(19) United States
(12) Patent Application Publication
(10) Pub. No.: US 2026/0056119 A1
(43) Pub. Date: Feb. 26, 2026

(54) IMPROVED METHOD FOR CLASSIFICATION OF AN EDIBLE SEED AND A SCANNING DEVICE THEREFOR

(71) Applicant: SURENUT PTY LTD, Brisbane, Queensland (AU)

(72) Inventors: Tom MARTIN, Brisbane, Queensland (AU); Wilmer ARIZA, Brisbane, Queensland (AU); Gayatri MISHRA, Brisbane, Queensland (AU)

(21) Appl. No.: 19/105,700

(22) PCT Filed: Aug. 23, 2023
(86) PCT No.: PCT/AU2023/050805

§ 371 Date: Feb. 21, 2025

(30) Foreign Application Priority Data
Aug. 24, 2022 (AU) 2022902420

(51) Int. Cl. G01N 21/31; B07C 5/342; G01N 33/02

(52) U.S. Cl. CPC G01N 21/314; B07C 5/3427; G01N 33/02; G01N 2021/3174

(57) ABSTRACT

A method for detecting an aflatoxin on a seed which includes determining wavelengths at which there is greatest difference in reflectance intensities of control and contaminated seed, and comparing reflectance intensities from the captured image with reflectance intensities indicative of an aflatoxin presence at a predetermined concentration. Seeds are ejected that have an aflatoxin concentration greater than the predetermined concentration as indicated by the reflectance intensities from the captured images. Examples of seeds include a nut or legume.

### FIELD OF INVENTION

The present disclosure relates to a method and system for classification of an edible grain seed using electromagnetic radiation. While an application of the present disclosure is in the classification of edible seeds, it could equally well be applied to the classification of any foodstuff for human and/or animal consumption.

### BACKGROUND OF INVENTION

Aflatoxin is a known carcinogen globally affecting over 25% of crops mainly tree nuts, peanuts, maize, cottonseed. Over 4 billion people worldwide are exposed to dietary aflatoxins. It is estimated that between 5-28% of liver cancers in humans could be attributed to aflatoxins. Every season, almond-processors detect out-of-specification product on receipt, which then must be diverted from sale for human consumption as there is no effective means of eliminating aflatoxins from almonds without significant damage to the product. This removal and reduction in potential return results in large losses.

Due to stringent food safety regulations, including a low contamination threshold for carcinogenic fungal aflatoxins, many countries require almond shipments to be certified. Industry standard processes for grading and testing harvested almonds are labour-intensive, time consuming, and expensive. With manual inspection, it is impossible to identify low levels of contaminants such as aflatoxins, which have very low tolerance in food products (below 5 ppb). In Australia and other major nut-producing countries, composition and safety evaluations are conducted using wet lab analyses. Wet chemical analysis for aflatoxin screening of almonds is destructive, slow, and expensive. Accordingly, there is a need for an alternative and automated almond classification method, which is rapid, objective, non-destructive, consistent, and accurate, and a scanning device useful in such a method.

Australia is the second largest producer of almonds after the United States of America. Australian almonds are exported both as kernel (71%) and inshell (29%). Most of the almond production in Australia is concentrated in the Riverland, Sunraysia and Riverina regions. The Australian almond industry contributes significantly to the local communities both socially and economically in almond growing and processing regions.

To deliver optimum product quality, almond kernels are sampled regularly (typically every one tonne bin) from the processing line and manually inspected to assign an appropriate commercial grade. However, manual sampling is labour intensive, slow and subjective which may lead to inconsistent and inaccurate grading. Also, food safety is increasingly becoming an important issue in the almond market like in other food commodities.

Due to stringent food safety regulations, including a low contamination threshold for carcinogenic fungal aflatoxins, many countries require almond shipments to be certified. Industry standard processes for grading and testing harvested almonds are labour-intensive, time consuming, and expensive. In Australia and other major nut producing countries, composition and safety evaluations are conducted using wet lab analyses. Depending on the season as much as 15% of the total Australian crop could be “out-of-spec” for mycotoxins. By detecting levels of aflatoxin early in the processing phase, informed decisions can be made-directing affected almonds to markets that require forms of heat treatment such as roasting and blanching, which can lower aflatoxin levels to allow entry to some markets.

The European Union presently enforces a regulation that limits the total aflatoxin content in ready-to-eat almonds to a maximum level of 8 μg kg⁻¹ (Commission Regulation (EC) No. 1881/2006 amended by Commission Regulation (EU) No. 165/2010), while the U.S. Food and Drug Administration (USFDA) maintains a 20 μg kg⁻¹ threshold. The distribution of aflatoxins in agricultural products has a high level of randomness because mould growth is dependent on many variables, including humidity, wind, shade, and other factors. The random distribution of aflatoxin in harvested crops is further increased due to multiple handling operations and processes, including partially or fully automated operations and processes. There are two primary existing methods for aflatoxin detection: liquid chromatography (LC) and UV fluorescence. Liquid chromatography is a destructive laboratory testing method useful for extracting and separating aflatoxins to determine an aflatoxin concentration of the input product. Since laboratory methods of detection require the sample to be ground up and chemically treated, in a sense destroying the product, test validation is limited. Therefore, the usefulness of the method to remove contaminated nuts from a larger quantity is limited. Moreover, diversity and abundance of aflatoxin demands higher number of samples need to be destroyed during analysis, which leads to wastage of the huge number of almonds.

Aflatoxin is fluorescent substance. Specifically, aflatoxin can be detected around a 435 nm wavelength when exposed to UV light at 365 nm. However, the whiter the substrate, the easier the recognition of aflatoxin from the image. The coloured skin of certain foodstuffs, such as the brown skin of almonds cancels the effect of fluorescence from aflatoxin and impedes the effective use of UV light for detection of aflatoxin. Additionally, the brown surface of almonds produces fluorescence response at high spectrum levels from any applied light source with a large shift of frequency. Therefore, what is needed is a rapid, non-destructive, reliable, and accurate detection and measurement of aflatoxin for the almond industry.

It will be clearly understood that, if a prior art publication is referred to herein, this reference does not constitute an admission that the publication forms part of the common general knowledge in the art in Australia or in any other country.

### SUMMARY

The present disclosure in one aspect sets out a method for classifying a foodstuff. The foodstuff may be an edible seed (including any grain, nut or legume). The method may include illuminating the edible seed with at least one wavelength of electromagnetic radiation, the at least one wavelength of electromagnetic radiation is partially reflected by each seed including specific signals from aflatoxin if it is present, detecting the reflected signal to provide a detected aflatoxin signal, comparing the detected aflatoxin signal with predetermined reflectance levels from known concentrations of aflatoxin to provide the first accurate measurement of aflatoxin concentrations, and classifying the edible seed aflatoxin concentration.

The present disclosure in another aspect sets out a scanning device for classifying a foodstuff. The foodstuff may be an edible seed, including any grain, nut or legume. The scanning device may include a reservoir configured to hold at least one edible seed, a chute, having at least one electromagnetic radiation source to illuminate the seed with at least three wavelengths of electromagnetic radiation, the at least three wavelengths of electromagnetic radiation causing a reflection signal of at least one type of aflatoxin, a camera configured to detect the reflected light, a filter to remove extraneous reflections, and to provide the aflatoxin signal; and a microprocessor configured to: compare the detected aflatoxin signal with predetermined signals of known concentration of the at least one type of aflatoxin to provide a first calibrated measurement of aflatoxin concentration, and classify the edible seed relative to the first measured aflatoxin concentration, accurate to +/−0.16 μg.

Foodstuff for human consumption and/or animal consumption must be kept dry and free from mould to reduce the risks associated with contamination. It is not always possible to eliminate the growth of mould on foodstuff and, accordingly, it would be beneficial to have a method of classifying the level of contamination of a foodstuff by a mould, for example Aspergillus sp.

Foodstuff for human consumption that may be contaminated by mould under inappropriate storage conditions includes edible seeds, cereals, fruits, and vegetables. Foodstuff for animal consumption that may be contaminated by mould under inappropriate storage conditions include hay, fodder, feed grains, animal feed pellets, animal feed crumbles, and animal feed mixes.

In another preferred embodiment, the present disclosure relates to a method for detecting an aflatoxin on a seed. The method includes sorting a plurality of seeds (including any grains, nuts or legumes) in a single file array; capturing a plurality of near or shortwave infrared images of each seed; comparing wavelengths from the captured image with wavelengths indicative of an aflatoxin presence at a predetermined concentration; and ejecting from the plurality of seeds those seeds that have an aflatoxin concentration greater than the predetermined concentration as indicated by the wavelengths from the captured images.

In one aspect, the captured images are pre-processed using the Savitzky-Golay 2nd (SG-2nd) derivative, to reveal some major differences in the reflectance intensities of the control and contaminated samples of almond kernels around 960 nm, 980 nm, 1050 nm, 1110 nm, 1150 nm, 1210 nm, 1250 nm, 1340 nm, 1390 nm, 1450 nm, and 1680 nm (feature wavelengths).

In another aspect, a competitive adaptive reweighted sampling (CARS) algorithm is used to select the feature wavelengths, removing the remaining redundant wavelengths from the model, for extraction of useful information in the shortest possible time, allowing for rapid quantification of AFB1 at commercial scale.

In a further aspect, reflectance of one or more control seed is measured at the feature wavelengths and compared to reflectance of the seed to be tested for contamination at those same feature wavelengths. As reflectance intensity increases with increase in the level of aflatoxin, this embodiment enables determination of the concentration (or level) of aflatoxin in the seed.

In yet a further aspect, multiple linear regression (MLR) models are used for quantification of aflatoxin concentration, to simplify the prediction process and to improve calculation speed. A formula used for the calculations may be:

$$Y = {β}_{0} + {\sum }_{i = 1}^{k} {β}_{i} {X}_{i} + {\sum }_{i = 1}^{k} {\sum }_{j = 1}^{k} {β}_{ij} {X}_{i} {X}_{j} + ϵ$$

where Y represents the aflatoxin concentration (ug/g); β₀ is the intercept; β₁ and βᵢⱼ are the linear and interactive coefficients; Xᵢ, Xⱼ are the reflectance values of the feature wavelengths and ε is error; k is the number of feature wavelengths used.

In another preferred embodiment, the present disclosure relates to a system for determining when a seed has an unsafe concentration of aflatoxin. The system includes a seed reservoir; a chute from the reservoir to a rotating glass disc; a plurality of light sources configured to emit a suppressed visible light upon each gain seed; and a plurality of hyperspectral or multi-spectral cameras configured to each capture a plurality of spectral images of each seed. The system also includes a processor configured to align and segment a spectral cube, determine an average reflectance for each spectral image, compare the average reflectance with a predetermined reflectance value indicative of a presence of an aflatoxin concentration designated to fail a predetermined health standard, and send instructions to a diverter to separate a seed determined to fail the health standard.

In one embodiment, filter wheels may be used to position a selected filter, or combination of filters, in the imaging path quickly and accurately. This may attenuate the light intensity or prevent unwanted spectral wavelengths from contaminating the image.

Because filter wheels move, it may be preferable to use a multi-spectral camera. What is needed is a low cost and high speed multi-spectral camera.

In one preferred embodiment the present disclosure relates to a multi-spectral imaging system having one or more camera, one or more lens, and one or more filter.

It is to be understood that both the foregoing general description and the following detailed description are exemplary and explanatory only and are not restrictive of the invention, as claimed. In the present specification and claims, the word “comprising” and its derivatives including “comprises” and “comprise” include each of the stated integers but does not exclude the inclusion of one or more further integers.

It will be appreciated that reference herein to “preferred” or “preferably” is intended as exemplary only.

The claims as filed and attached with this specification are hereby incorporated by reference into the text of the present description. The accompanying drawings, which are incorporated in and constitute a part of this specification, illustrate several embodiments of the invention and together with the description, explain the principles of the invention.

### BRIEF DESCRIPTION OF THE DRAWINGS

FIG. 1 is a top view of a scanning device in accordance with a preferred embodiment of the present disclosure.

FIG. 2 is a front view of the scanning device of FIG. 1.

FIG. 3 is another side view of the scanning device of FIG. 1.

FIG. 4 is a bottom view of the scanning device of FIG. 1.

FIG. 5 is a perspective view of the scanning device of FIG. 1.

FIG. 6 is an image comparing spectral radiant flux with wavelength.

FIG. 7 is a chart demonstrating the feature wavelength selection by CARS for AFB1 prediction.

FIG. 8 (a) is a chart demonstrating mean reflectance of spectra obtained from control and aflatoxin infected almond kernels.

FIG. 8 (b) is a chart demonstrating mean reflectance of SG-2nd derivative pre-processed spectra obtained from control and aflatoxin infected almond kernels.

FIG. 9 is a multi-spectral system with a spinning wheel.

FIG. 10 is a NIR light system.

### DETAILED DESCRIPTION

Reference will now be made in detail to the present preferred embodiments of the invention, examples of which are illustrated in the accompanying drawings.

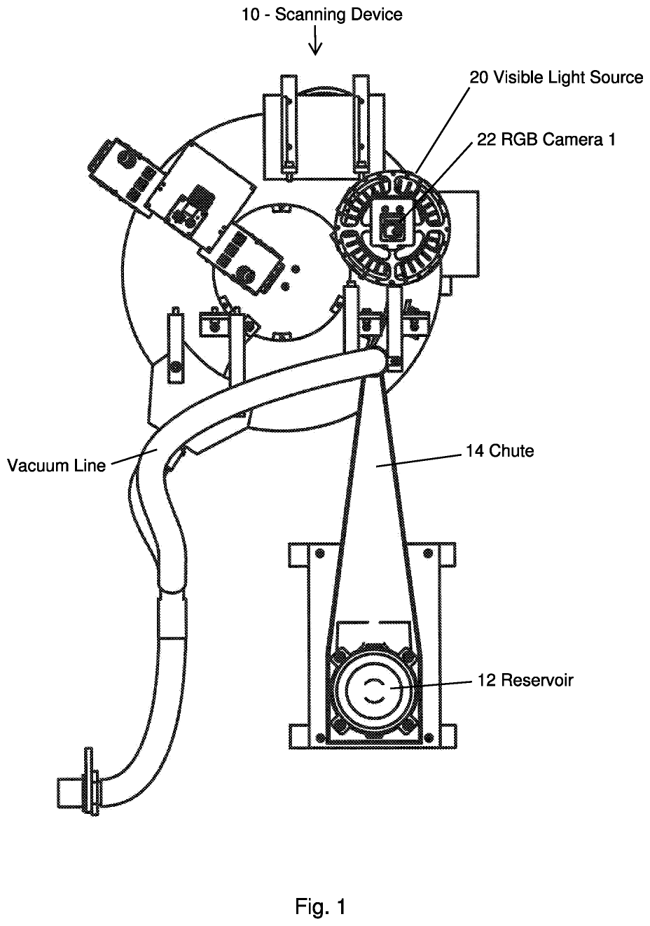

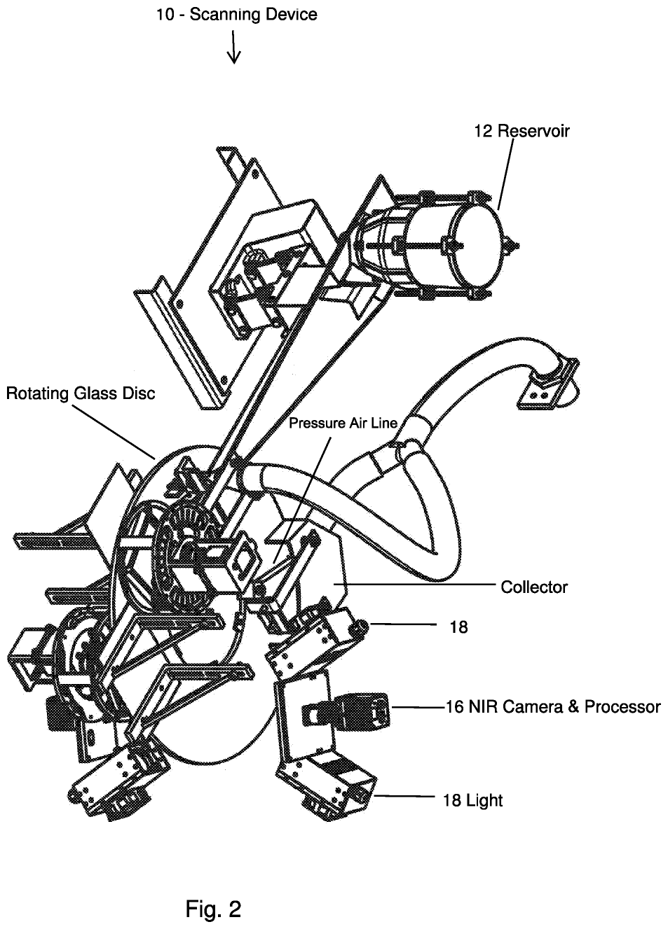

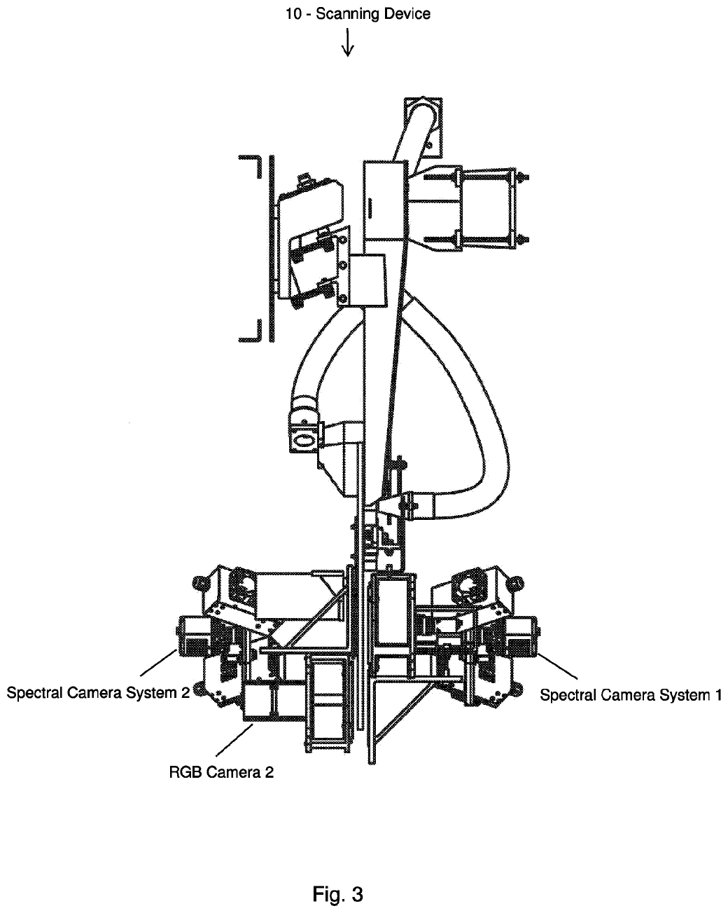

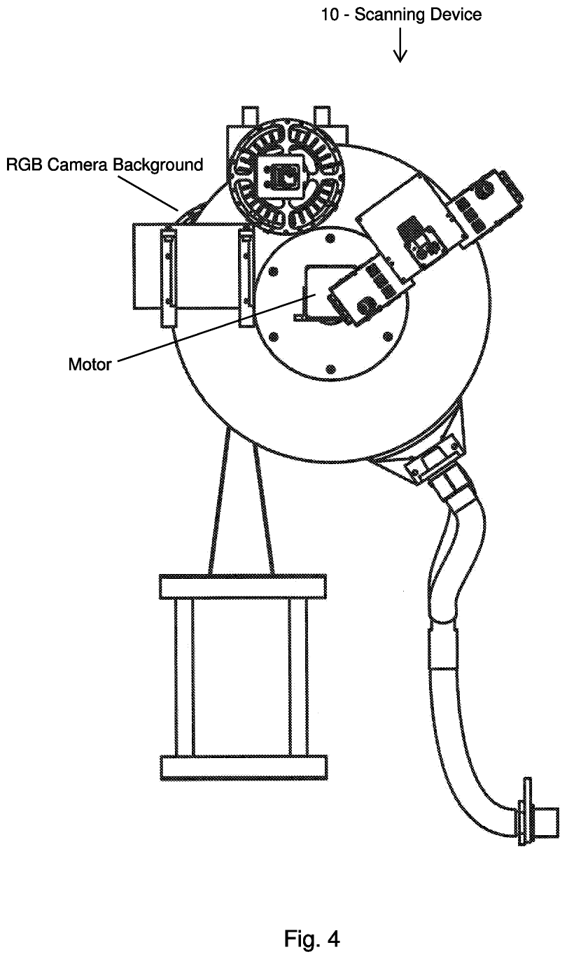

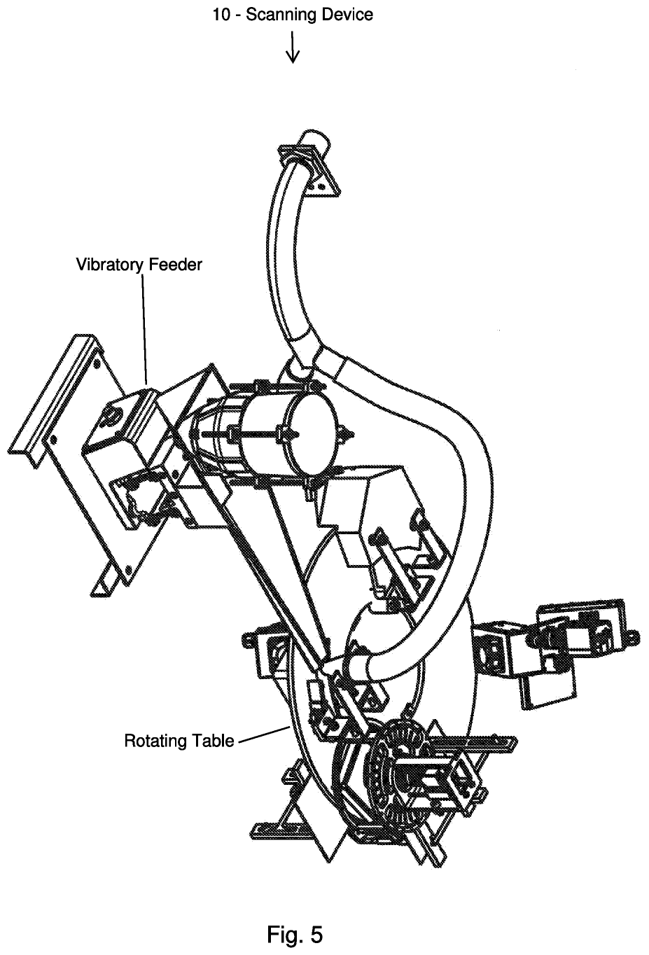

FIGS. 1 to 5 show a preferred embodiment of a scanning device 10. The preferred elements of scanning device 10 and their interrelationships are described below. Scanning device 10 includes a reservoir 12, a chute 14, a near infrared (NIR) camera detector 16, and a microprocessor. Reservoir 12 is configured to hold at least one edible seed. Scanning device 10 has at least one electromagnetic radiation source 18. In use, the electromagnetic radiation source 18 illuminates the edible seed with at least one wavelength of electromagnetic radiation. At specific wavelengths of NIR radiation, the reflectance can be measured and after calibration the detection of at least one type of aflatoxin can be accurately measured. An NIR camera 16 is configured to capture any reflected light. The microprocessor is configured to compare the various wavelengths of this reflectance and accurately measure any levels of aflatoxin that may be present by comparison with previous calibrations from accurately laboratory dosed specimens. A signal detector 16 is a near infrared (NIR) hyperspectral or multi-spectral camera. Near infrared (NIR) hyperspectral or multi-spectral cameras can detect internal defects which are not visible to human eyes or to cameras operating in the visible region of the electromagnetic spectrum. This enables the detection and classification of aflatoxins.

Hyperspectral imaging acquires images with hundreds of continuous wave bands usually by the application of a spectrograph and a sensitive area detector whereas for multi-spectral imaging a sensitive area detector is usually paired with a or series of specific waveband filters or a waveband tunable light source. The result of both systems is called a data cube. This data cube is composed of a M×N×W matrix, where M and N are the position data and W is the waveband value.

Scanning device 10 further includes a visible light source 20 and a visible light detector 22. Visible light source 20 illuminates the edible seed and visible light detector 22 detects an image of the edible seed. In a preferred embodiment, visible light source 20 is a halogen light source. The microprocessor is configured to detect a blemish on a surface of the edible seed as shown in the image, compare the blemish to a predetermined blemish signal of a known concentration of the aflatoxin to provide a second estimated aflatoxin concentration, and classify the edible seed relative to the second estimated aflatoxin concentration.

A multiple linear regression (MLR) model may be used for quantification of aflatoxin concentration, to simplify the prediction process and to improve calculation speed. A formula used for the calculations may be:

$$Y = {β}_{0} + {\sum }_{i = 1}^{k} {β}_{i} {X}_{i} + {\sum }_{i = 1}^{k} {\sum }_{j = 1}^{k} {β}_{ij} {X}_{i} {X}_{j} + ϵ$$

where Y represents the aflatoxin concentration (ug/g); β₀ is the intercept; βᵢ and βᵢⱼ are the linear and interactive coefficients; Xᵢ, Xⱼ are the reflectance values of the feature wavelengths and ε is error; k is the number of feature wavelengths used.

Preferably, the image has a dimension approximately four times the width of the edible seed. Further preferably, visible light detector 22 is an RGB light camera. In a further preferred embodiment, scanning device 10 includes a NIR spectrophotometer.

RGB cameras are used on each side of the glass plate and NIR cameras, from both sides also. Many wavelengths may be used depending upon the characteristic being investigated, for example aflatoxin, FFA, PV, and moisture.

Moisture content, rancidity—free fatty acids (FFA) and peroxide value (PV) are good quality indicators of almonds and industry use this data to inform product shelf life. Rancidity and aflatoxin have significant effect on consumer health. The four major and naturally known aflatoxins are produced by the Aspergillus species of mould. Aflatoxin B₁ (AFB1), aflatoxin B₂ (AFB2), aflatoxin G₁ (AFG1) and aflatoxin G₂ (AFG2) are predominantly encountered, with AFB1 being the most common and the most potent genotoxic and carcinogenic of the aflatoxins. Detectable aflatoxins can be produced by Aspergillus flavus or Aspergillus parasiticus.

The edible seed may be any grain, nut or legume, for example only, an almond, a Brazil nut, a candlenut, a cashew, a Chilean hazelnut, a hazelnut, a macadamia, a peanut, a pecan, a pine nut, a pistachio, a walnut, maize, cottonseed or any other cereal, oil seed, pulse, peanut or tree nut.

It will be appreciated that the scanning device 10 and the reservoir 12 may be configurable to accommodate any foodstuff suitable for human and/or animal consumption. A plurality of images of each edible seed is compiled, preferably using a virtual cube, to provide a composite 3D representation of the edible seed. Artificial intelligence, i.e., deep learning, including forward selection and backward elimination, stepwise selection, etc., is employed to discern all fault parameters of the edible seed. Where more than one fault is present on an edible seed, the suitability determination is determined by what is least desirable for the final consumer to experience/taste—that is fault parameters are ranked.

The edible seeds can be sorted to achieve desired specifications for the edible seeds, e.g., edible seeds can be categorised for human or animal consumption, for meal, crushed nuts, or nut milk production. The data produced from this analyser determination informs down the line managers in sorting, processing and marketing. If desired, the system can be linked to digital sorting machines to enhance performance to be focused only on customer specifications.

The scanning device can be connected to a network or the internet for online management and/or reporting. The scanning device may include at least one glass plate for the edible seed to be supported and/or conveyed across. The scanning device may include a mechanism to clean the glass plate. Alternatively, the glass plate may be electrically charged to repel dust particles. It will be appreciated that a dust particle may include a fibre, particulate organic material, or the like. Such particulate organic material may originate from the edible seed.

Referring to FIGS. 1 to 5, a preferred method for classifying an edible seed is described below. The method includes illuminating the edible seed with a number of wavelengths of NIR electromagnetic radiation. The reflectance of electromagnetic radiation from the seed is then analysed and compared to the reflectance generated by seeds dosed at known rates of aflatoxin in a laboratory. The calibration used is preferably measuring Aflatoxin B₁ to +/−0.16 μg/g.

Preferably, the wavelength of electromagnetic radiation is near infrared radiation.

The specific wavelength used will have an impact on the accuracy and speed of the results, allowing for rapid quantification of AFB1 at commercial scale. In almond kernels, for example, major differences in the reflectance intensities of control and contaminated kernels occur at around 960 nm, 980 nm, 1050 nm, 1110 nm, 1150 nm, 1210 nm, 1250 nm, 1340 nm, 1390 nm, 1450 nm, and 1680 nm, making these wavelengths the preferred (or feature) wavelengths to use for testing almond kernels. Reflectance of one or more control seed is measured at the feature wavelengths, and compared to reflectance of the seed to be tested for contamination at those same feature wavelengths. As reflectance intensity increases with increase in the level of aflatoxin, this enables determination of the concentration (or level) of aflatoxin in the seed.

The feature wavelengths may be determined by pre-processing captured images using the Savitzky-Golay 2nd (SG-2nd) derivative, to reveal any major differences in the reflectance intensities of the control and contaminated samples of edible seed.

The feature wavelengths may be determined by a competitive adaptive reweighted sampling (CARS) algorithm, removing the remaining redundant wavelengths from the model, for extraction of useful information in the shortest possible time.

The aflatoxin may be produced by Aspergillus sp. More particularly, the aflatoxin may be produced by Aspergillus flavus Aspergillus parasiticus. Preferably, the detectable aflatoxin is at least one of aflatoxin B₁, aflatoxin B₂, aflatoxin G₁, or aflatoxin G₂.

It will be appreciated that the steps described above may be performed in a different order, varied, or some steps omitted entirely without departing from the scope of the present invention.

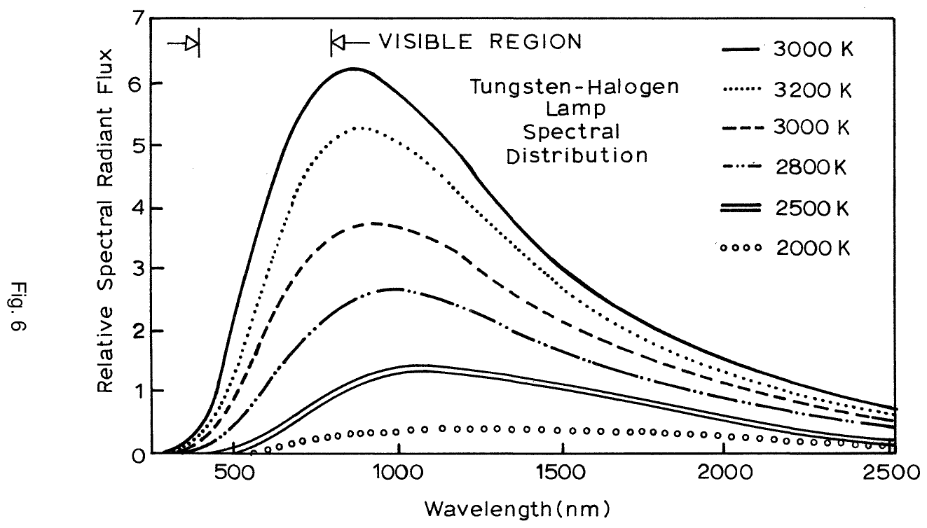

FIG. 6 shows a full spectrum, with visible NIR range as indicated. Hyperspectral imaging is a combination of both spatial imaging and spectroscopy (spectral). The hyperspectral images are three-dimensional arrays (m×n×λ), where m and n are the spatial axes and λ is the spectral information. Hyperspectral cameras are used with broadband halogen lights that produce the light source in full spectrum. Objects subjected to high values of light will produce light noise at higher range of the spectrum. This noise carries colour information that obscure chemical information. To help overcome this, a halogen light only at the higher range is produced by the subtraction of lower range light.

Filter wheels may be used to position a selected filter, or combination of filters, in the imaging path quickly and accurately. This may attenuate the light intensity or prevent unwanted spectral wavelengths from contaminating the image.

For some filter wheels, a hyperspectral camera cannot be used as the speed of the filter wheel means that the camera is required to scan at a speed of over 20 data cubes per second. At this speed, hyperspectral cameras will show a deformed image as the object rotates in front of the camera and even after correcting the pixel position by curvature the mean spectrum of the product will suffer distortions. In such cases, a multi-spectral camera may be used.

Not all multi-spectral cameras will be suitable. Some commercial grade multi-spectral cameras such as the SpectroCam from Ocean Insight can only take two to four data cubes per second. Other commercial grade equipment such as the MS-IR from Telops has a high rate of frames per second and can accommodate up to 8 filters, but the cost of deployment can be up to twice the cost of a commercially available area scan hyperspectral camera.

The present disclosure includes a high speed multispectral system that is capable of replacing a hyperspectral camera in a production line and can be designed and prototyped with lower cost than an equivalent hyperspectral or commercially available multispectral system.

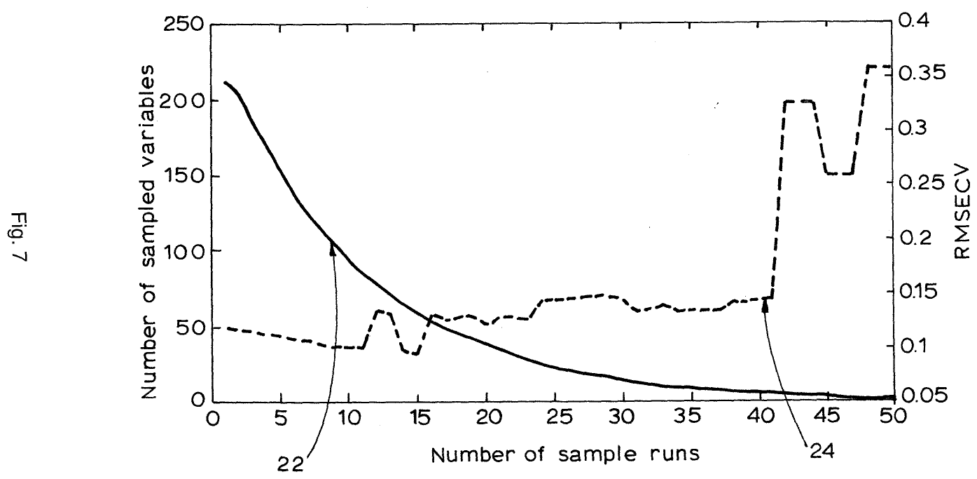

FIG. 7 shows the feature wavelength selection for AFB1. The line start starts at above 200 sampled variables 22 indicates the selected wavelengths, which reduces as the sampling increases. The line that starts at about 50 sampled variables 24 shows the variation of root mean square error of cross-validation (RMSECV) values with the number of sampling. As the elimination process of redundant wavelengths was increased, RMSECV values 24 were first reduced moderately and then gradually increased with the elimination of feature wavelengths. The optimum wavelengths were chosen based on the minimum RMSECV values 24. For AFB1 calibration set, the RMSECV value 24 was lowest at the 15th sampling and the corresponding number of feature wavelengths was 58.

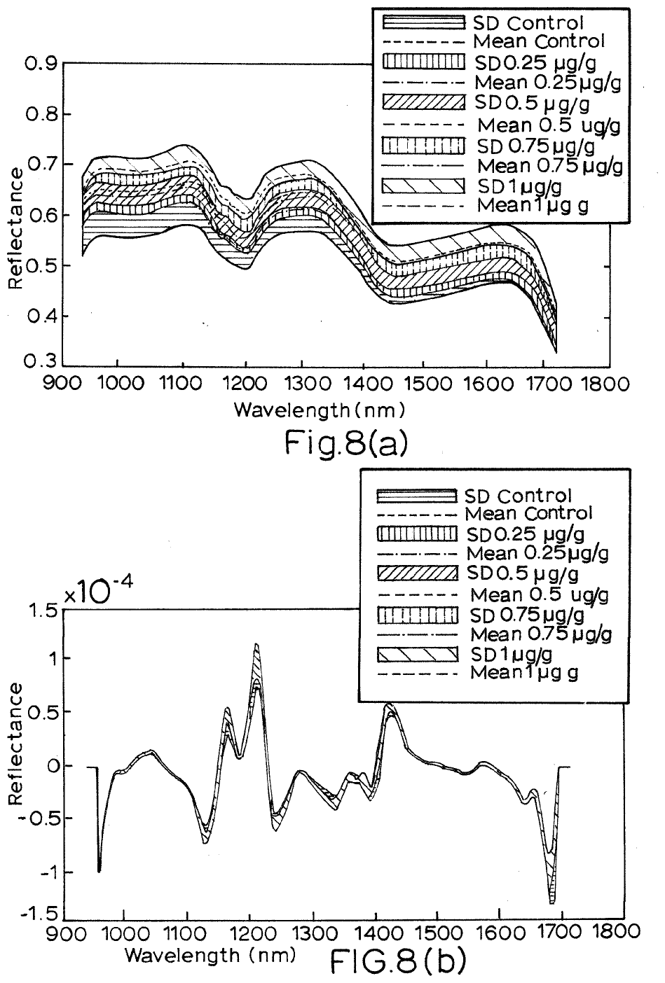

FIG. 8 (a) shows the mean reflectance of spectra obtained from control and contaminated samples of almond kernels. The shape of the spectral curves of all samples were similar, indicating similarity in the chemical composition of almond kernels. However, while comparing the mean spectra, it was found that at certain wavelengths, reflectance of the control kernels was lower as compared to the contaminated kernels and the reflectance intensity increased with increase in the level of the aflatoxin.

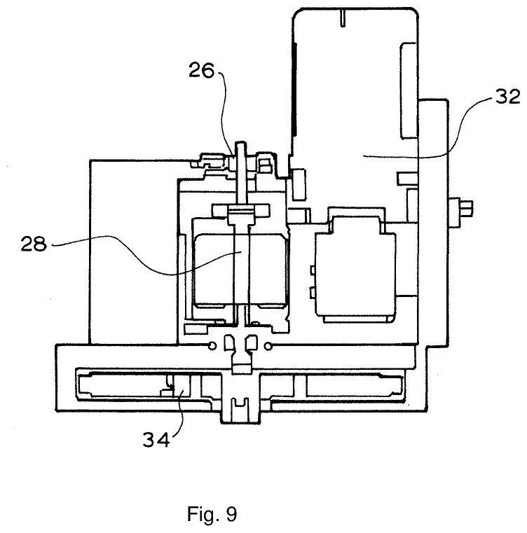

FIG. 8 (b) shows the mean reflectance of Savitzky-Golay 2nd derivative pre-processed spectra obtained from control and contaminated samples of almond kernels. The shape of the spectral curves reveals some major differences in the reflectance intensities of the control and contaminated samples of almond kernels around 960 nm, 980 nm, 1050 nm, 1110 nm, 1150 nm, 1210 nm, 1250 nm, 1340 nm, 1390 nm, 1450 nm, and 1680 nm. FIG. 9 is a multi-spectral camera system with a filter wheel 34.

Filter wheel 34 may include a wheel and filters. In a preferred embodiment, there may be 8 filters. These may be NIR filters, of 32 mm.

Filter wheel 34 may be rotated by a motor 28. Motor 28 may be a SC040A servomotor as it has a PID controller for the motor RPM and can be precisely positioned to capture the required images. The terms SC040A, servomotor, and PID would be understood by a person skilled in the art.

Filter wheel 34 may have a permanent rotation at specific RPM with high accuracy, and a position or absolute encoder 26 which measures the current location of the filter wheel 34 and overrides any microcontroller (MCU) in camera to trigger filter wheel 34 when the desired filter is in position, and two pure NIR lights 32 to provide the correct illumination.

The angle region in which each filter will be on view, encoder pulses and minimum integration time for the camera may be calculated by:

$$View Angle = \frac{360 °}{2 \times π \times r} \times Δ F \qquad ( 1 )$$

$$Pulse region = \frac{360 °}{Encoder pulses} \times View Angle \qquad ( 2 )$$

$$I ( t ) = \frac{View Angle}{RPS * 360 °} \qquad ( 3 )$$

where r is the radius from the filter location to motor 28, ΔF is the sum of the giving space at each side of the filter, Encoder pulses are the number of division available for each revolution of encoder 26, and RPS is the revolution per second of the system.

The pseudocode for triggering of the multispectral camera may be in according with the algorithm:

| Result: Multispectral Cube | | | | |
|---|---|---|---|---|
| initialization of camera; | | | | |
| while While do | | | | |
| \| | if Trigger Change to High then | | | |
| \| | \| | Record time start; | | |
| \| | else if Trigger Change to Low then | | | |
| \| | \| | Calculate time between; | | |
| \| | \| | if Time less than desire delay then | | |
| \| | \| | \| | Delay[i]=Time between; | |
| \| | \| | else | | |
| \| | \| | \| | Delay[i]=Desire delay; | |
| \| | \| | end | | |
| \| | end | | | |
| \| | if Delay[1]>0 then | | | |
| \| | \| | for j=1:n do | | |
| \| | \| | \| | if Current Time>Delay[1] & Encoder | |
| \| | \| | \| | position j then | |
| \| | \| | \| | \| | Trigger On; |
| \| | \| | \| | \| | delay(Time on); |
| \| | \| | \| | \| | Trigger Off; |
| \| | \| | \| | \| | delay(Time Off); |
| \| | \| | \| | \| | if Encoder position≈n then |
| \| | \| | \| | \| | \| Delay<<; |
| \| | \| | \| | \| | end |

| | | |
|---|---|---|
| \| | \| | end |

end

The first section calculates the delay time of an incoming trigger signal and stores it in a matrix of delays. The second section executes the delays stored in the matrix of delays depending on the current time and filter wheel 34 position. The pseudocode is designed to always start from first filter position and produce 8 images per cycle.

The pseudocode may be written in C for microcontrollers Microchip ATmega328P running at 16 MHz.

The integration between the photoelectric switch and the trigger delay MCU may be done with a 4n25 optocoupler for DC systems. The use of the optocoupler is to reduce the possibility of damage to the camera by short circuits in the system.

Preferably, the multi-spectral camera system will work in the range of 900 nm to 1700 nm, scan at a speed of at least 20 data cubes per second, synchronise with other cameras in the system, scan a range of 1 to 8, for example, 6 wavebands, enable identification of the data in each waveband, order the data captured, and allow the correct illumination of the area to be scanned.

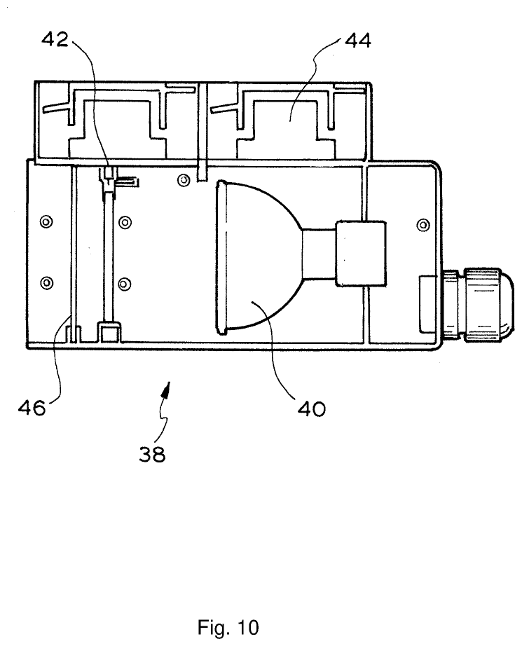

FIG. 10 is a NIR light system. Each NIR light 38 may be composed of a Quartz tungsten halogen light 40, a Quartz clear lens 42 and a high pass filter 46. Halogen light 40 produces almost homogeneous light in the range between 400-2500 nm. This light source however emits radiation and heat. Quartz clear lens 42 is placed in front of the lamp to reduce the heat against high pass filter 46. Heat may also be reduced by a cooling fan 44. Quartz clear lens 42 may be placed at 1 mm from the highest element of filter wheel 34. Finally, high pass filter 46 removes the visible light components. High pass filter 46 may be located at a minimum radius of 100 mm.

To help illustrate features of the method and system, several experiments are set forth below.

### EXPERIMENTAL METHODOLOGY

### (a) Aflatoxin Infection on Almonds

Nonpareil variety of healthy almonds kernels were selected as samples which have good appearance and uniform size. Standard aflatoxin B₁ solutions of various concentrations ranging from 5 μg/mL to 50 μg/mL were prepared by diluting it with 50:50 methanol and water. 20 μL drops of aflatoxin solution was added onto the almond surface using a pipette to prepare almond samples each containing 0.1 μg/g to 1 μg/g of aflatoxin B₁ (considering the average weight of single almond is 1 g).

The samples were divided into four groups, namely 2.5, 5,7.5 and 10 μg/g groups and healthy, noninfected almonds were considered as control for the experiments. The aflatoxin infested almonds were dried under natural air for drying of the methanol and water solution completely. Images of single almonds were scanned using a hyperspectral camera in the wavelength range of 900-1700 nm, one by one. Using 224 number of images of one almond at various wavelengths, the hyperspectral camera synthesized a hyperspectral image cube. The average spectra of each image were extracted after background removal and library of 3000 spectra was constructed. After capturing the images, the almond samples were destroyed, and aflatoxin content was verified using High Performance Liquid Chromatology (HPLC).

### (b) Moisture Content Analysis

Nonpareil variety of healthy almonds kernels were selected as samples which have good appearance and uniform size. The sample bulk was divided into four groups. A measured amount of distilled water was added to the almonds to bring the moisture content from 6% to 12%. Almonds with 4% moisture content were used as control sample for the experiment. The moisture conditioned samples were stored in the refrigerator for 7 days to reach equilibrium. After 7 days, about 3000 images of various moisture content almonds were scanned and spectral library was generated. After capturing the images, all the almonds were verified for the actual moisture content using oven drying method.

### (c) FFA and PV Value Analysis

Nonpareil variety of healthy almond bulk was divided into two groups, stored under 40° C. and 75% relative humidity for 1.5 months and 3 months, respectively to produce rancid almonds with higher FFA and PV value. Healthy almonds were used as control for the experiments. About 3000 images of various moisture content almonds were scanned and spectral library was generated. After capturing the images, all the almonds were verified for the actual FFA and PV content using standard titration method.

### (d) Selection of Specific Wavelengths and Model Development

About 3000 images including the healthy and aflatoxin treated almonds of each parameter were captured for model development. The reference analysis data for moisture, FFA, PV, aflatoxin data and spectral data from the hyperspectral camera were used to develop a Partial Least Square Regression model (PLSR) to predict the respective quality parameters of the single almond. PLSR model assigned a weight to each of the 224 number of wavelengths based on predictability of aflatoxin content. Hyperspectral data coupled with a full spectrum PLSR model yielded best prediction results. Few wavelengths with higher weights were selected and used for training of the model again to check the accuracy. Validation of the model using the selected number of wavelengths was done against the sample outside the training data. The PLSR model may follow the equation:

$$X - {TP}^{T} + E \\ Y = {UQ}^{T} + F \\ T = {XW}_{k}$$

where X is an n×m matrix of predictors, indicating n is the number of samples and m is the number of wavelengths; Y is an n×1 matrix of response variable; T is the score matrix of wavelengths, P is the m×k matrix of X loadings and Q is (1×k) matrix of Y loadings (k is the number of latent variables); Y is the reference data (n×1) to be predicted from X. E and F stand for random errors in X and Y, respectively, while W is PLS weights, and Wₖ is the m×k matrix of X weights. The association between spectra matrix (X) and quality attribute (Y) may be predicted using equations:

$$\hat{Y} = {XW}_{k} β + E \\ β = {W}_{k} \times {Q}^{T} = {W ( {P}^{T} W )}^{- 1} {Q}^{T}$$

where β is m×1 matrix containing the regression coefficients, obtained by the PLSR model equations. Ŷ is the predicted value of the response of interest.

The ideal number of latent variables is decided by cross validation when the root mean square error of cross-validation (RMSECV) reaches a minimum. The RMSECV is preferably calculated by the equations:

$${R}^{2} = 1 - \frac{{\sum }_{i = 1}^{n} {( {Y}_{i} - \hat{Y} )}^{2}}{{\sum }_{i = 1}^{n} {( {Y}_{i} - \hat{Y} )}^{2}} \\ RMSE = \sqrt{{\sum }_{i = 1}^{n} \frac{{( {Y}_{1} - Y )}^{2}}{n}}$$

### (e) Final Multispectral Imaging System Construction:

A near infrared (NIR) light was developed by the suppression of visible light from a halogen light source. Two NIR multispectral camera with specific number of frequencies to capture a series of spectral images to assemble a spectral cube. A rotating glass system presents single almonds to the capture system, one at a time. Almonds flowing in single file trigger a light sensor, the advance spectral camera system acquires multiple times for each almond and converted to a series of short pulses for each wavelength required to capture. The cube capture is aligned and segmented to extract the corresponded frequencies for the respective almond in the frame. The average reflectance for each spectrum is calculated. The average spectrums of the wavelengths used (or displayed) to calculate the moisture, FFA, PV and aflatoxin content of a single almond.

The system and method described herein has many benefits and advantages. For example only, features of one or more embodiments described herein can generate an almond colour index, and even discern one or more of scratches, chips, stains, embedded shells, insect damage, mould, and other characteristics on almonds that could affect the quality of the almond.

The features described with respect to one embodiment may be applied to other embodiments or combined with or interchanged with the features of other embodiments, as appropriate, without departing from the scope of the present invention.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

U.S. Pat. No. 10,021,369 is hereby incorporated by reference herein in its entirety.

### Definitions

Aflatoxins are poisonous carcinogens and mutagens that are produced by certain moulds (Aspergillus flavus and Aspergillus parasiticus) which grow in soil, decaying vegetation, hay, and grains.
Rancidity is the process of complete or incomplete oxidation or hydrolysis of moisture and/or oils when exposed to air, light, or humidity, resulting in unpleasant taste and odour. Pathways for rancidification include hydrolytic rancidity and oxidative rancidity.
Free fatty acids (FFA) are the by-product of hydrolytic rancidity, and it is one of the parameters used for measuring rancidity in almonds.
Peroxide value (PV) are the by-products of oxidative rancidity. It is one of the parameters used for measuring rancidity in almonds.
NIR light is reflective light; it bounces off objects much like visible light. Near infrared (NIR) light is typically defined as light in the 0.9-1.7 μm wavelength range but can also be classified from 0.7-2.5 μm. NIR images are not in colour, making objects composition easily recognisable.
Spectral cube is a group of spectral images in which each layer is composed of an image taken in a specific wavelength. Like an RGB image where each layer of the three images represent the wavelengths of red, green, blue. Spectral cubes are composed of higher number of layers.
Spectra is the representation of the reflectance values of an image at various wavelengths.
Multispectral camera captures image data within few specific wavelength ranges across the electromagnetic spectrum.

1-44. (canceled)

45. A method for classifying an edible seed, comprising:
illuminating the edible seed with at least one wavelength of electromagnetic radiation, wherein the at least one wavelength of electromagnetic radiation is partially reflected by the seed including one or more specific signal from aflatoxin if it is present and selected from the group consisting of 960 nm, 980 nm, 1050 nm, 1110 nm, 1150 nm, 1210 nm, 1250 nm, 1340 nm, 1390 nm, 1450 nm, and 1680 nm,
detecting the reflected signal to provide a detected aflatoxin signal,
comparing the detected aflatoxin signal with a predetermined signal from known concentrations of aflatoxin to provide a first accurate measurement of aflatoxin concentration, and
classifying the edible seed aflatoxin concentration.

46. The method of claim 45, wherein the images are captured with a hyperspectral camera.

47. The method of claim 45, wherein the images are captured with a multi-spectral camera.

48. The method of claim 45, wherein Savitzky Golay second derivative (SG-2nd) is used to pre-process spectral data to determine the wavelengths at which there is greatest difference in the reflectance intensities of control and contaminated seed.

49. The method of claim 45, wherein competitive adaptive reweighted sampling (CARS) algorithm, is used to remove redundant wavelengths.

50. The method of claim 45, wherein the wavelengths indicative of an aflatoxin presence are selected using one or more filter wheel.

51. The method of claim 50, wherein each filter wheel comprises:
a wheel;
one or more filters;
a rotation at specific RPM with high accuracy;
a position encoder which measures current location of the wheel and triggers the filter wheel when the desired filter is in position; and
one or more pure NIR lights.

52. The method of claim 51, wherein each NIR light comprises:
one or more Quartz tungsten halogen light;
one or more Quartz clear lens; and
one or more high pass filter.

53. The method claim 45, wherein the seed is a grain, nut, legume, almond kernel or peanut.

54. The method of claim 45, wherein the aflatoxin is Aflatoxin B₁.

55. A system for determining when a seed has a concentration of aflatoxin above a threshold value, comprising:
a seed reservoir;
a chute from said reservoir to a rotating glass disc;
a plurality of light sources configured to emit a light in a wavelength range of 900 nm to 1,700 nm;
a plurality of cameras configured to each capture a plurality of spectral images of each seed at one or more wavelength selected from the group consisting of 960 nm, 980 nm, 1050 nm, 1110 nm, 1150 nm, 1210 nm, 1250 nm, 1340 nm, 1390 nm, 1450 nm, and 1680 nm; and
a processor configured to align and segment a spectral cube, determine an average reflectance for each spectral image, compare the average reflectance with a predetermined reflectance threshold value indicative of a presence of an aflatoxin concentration designated to fail a predetermined health standard, and send instructions to a diverter to separate a seed determined to be above the threshold value.

56. The system of claim 55, wherein one or more of the cameras are hyperspectral cameras.

57. The system of claim 55, wherein one or more of the cameras are multi-spectral cameras.

58. The system of claim 55, further comprising a rotating glass plate to present a plurality of the seeds in a single file array.

59. The system of claim 55, further comprising one or more filter wheel.

60. The system of claim 59, wherein each filter wheel comprises:
a wheel;
one or more filters;
a rotation at specific RPM with high accuracy;
a position encoder which measures current location of the wheel and triggers the filter wheel when the desired filter is in position; and
one or more pure NIR lights.

61. The system of claim 55, wherein seed is a grain, nut, legume, almond kernel or peanut.

62. The system of claim 55, wherein the aflatoxin is Aflatoxin B₁.

63. A scanning device for classifying an aflatoxin concentration of at least one edible seed comprising:
a reservoir configured to hold the at least one edible seed,
a chute,
at least one electromagnetic radiation source to illuminate the seed with at least three wavelengths of electromagnetic radiation, the at least three wavelengths of electromagnetic radiation causing a reflection signal of at least one type of aflatoxin,
a camera configured to detect the reflected light at three or more wavelengths selected from the group consisting of 960 nm, 980 nm, 1050 nm, 1110 nm, 1150 nm, 1210 nm, 1250 nm, 1340 nm, 1390 nm, 1450 nm, and 1680 nm,
a filter to remove extraneous reflections, and to provide the aflatoxin signal at the three or more wavelengths selected from the group consisting of 960 nm, 980 nm, 1050 nm, 1110 nm, 1150 nm, 1210 nm, 1250 nm, 1340 nm, 1390 nm, 1450 nm, and 1680 nm; and
a microprocessor configured to: compare the detected aflatoxin signal with predetermined signals of known concentration of the at least one type of aflatoxin to provide a first calibrated measurement of aflatoxin concentration, and classify the edible seed relative to the first measured aflatoxin concentration, accurate to +/−0.16 μg.
